(19) United States
(12) Patent Application Publication
(10) Pub. No.: US 2016/0213645 A1
(43) Pub. Date: Jul. 28, 2016

(54) COMBINATION OF LEVETIRACETAM AND A BETA-SECRETASE (BACE) INHIBITOR

(71) Applicant: James Wallace, Greenwich, CT (US)

(72) Inventor: James Wallace, Greenwich, CT (US)

(21) Appl. No.: 15/089,563

(22) Filed: Apr. 3, 2016

Related U.S. Application Data
(60) Provisional application No. 62/143,057, filed on Apr. 4, 2015.

(51) Int. Cl. A61K 31/4015; A61K 45/06

(52) U.S. Cl. CPC A61K 31/4015; A61K 45/06

(57) ABSTRACT

This invention is a pharmaceutical composition for the treatment and prevention of disease comprising of levetiracetam, as an active ingredient, to stabilize intracellular calcium ion concentrations, and a beta-secretase inhibitor, as an active ingredient, to decrease beta-secretase activity.

### CROSS-REFERENCE TO RELATED APPLICATIONS

This present application claims the benefit of U.S. Provisional Application No. 62/143,057 filed on Apr. 4, 2015.

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM, LISTING COMPACT DISC APPENDIX

Not Applicable

### BACKGROUND

Derangements in intracellular calcium ion signaling have been characterized as an early, or a proximal occurrence in disease, including neurodegenerative disease such as Alzheimer's disease (see Berridge M. J. et al., Nature, vol. 395, 1998, 645-648; Stutzmann G. E., Neuroscientist, vol. 13, 2007, 546-559; Lopez J. R. et al., J. Neurochem., vol. 105, 2008, 262-271; Bezprozvanny I. and Mattson M. P., Trends Neurosci., vol. 31, 2008, 454-463; Berridge M. J., Neurochem. Res., vol. 36, 2011, 1149-1156; Chakroborty S. et al., Sci. China Life Sci., vol. 54, 2011, 752-762; De Caluwe J., Dupont G., J. Theor. Biol., vol. 331, 2013, 12-18; Bezprozvanny I. and Hiesinger P. R., Mol. Neurodegener., vol. 8, 2013, 23; Wallace J., Cell Calcium, vol. 55, 2014, 175-181).

It is generally accepted that Alzheimer's disease is characterized by the formation and deposition of amyloid-beta (“Aβ” or “Abets”) and neurofibrillary tangles in brain tissue. It is also generally accepted that amyloid-beta formation precedes neurofibrillary tangle formation, and that amyloid-beta formation results from the abnormal cleavage of amyloid precursor protein, a brain protein, by a two-step process that involves cleavage by a beta-secretase, and then a gamma-secretase, with beta-secretase cleavage being the proximal event to gamma-secretase cleavage (see Hardy J. and Allsop D., Trends Pharmacol. Sci., vol. 12, 1991, 383-388; Cole S. L. and Vassar R., Mol. Neurodegener., vol. 2, 2007, 22; Ghosh A. K. et al., Neurotherapeutics, vol. 5, 2008, 399-408; Querfurth H. W. and LaFerla F. M., N. Engl. J. Med., vol. 362, 2010, 329-344; Vassar R. et al., J. Neurochem., vol. 130, 2014, 4-28).

Thus, two processes of significance in disease are (1) intracellular calcium ion signaling derangements and (2) the cleavage of amyloid precursor protein by beta-secretase.

A pharmaceutical composition comprising of at least two active ingredients, one active ingredient that modulates intracellular calcium ion signaling, and one active ingredient that inhibits beta-secretase activity; may be useful for the treatment or prevention of disease characterized by derangements of intracellular calcium ion signaling and the abnormal cleavage of amyloid precursor protein by beta-secretase, such as Alzheimer's disease.

### SUMMARY OF THE INVENTION

This invention relates to the field of pharmacology. Specifically, a pharmaceutical composition comprising of at least two active ingredients: (1) levetiracetam, as an active ingredient, to modulate intracellular calcium ion signaling and (2) a beta-secretase inhibitor, as an active ingredient, to decrease beta-secretase cleavage of amyloid precursor protein; for the treatment and/or the prevention of conditions or diseases in which the stabilization of intracellular calcium ion derangements and the inhibition of beta-secretase activity is desired, including Alzheimer's disease. This invention is also directed to the treatment of non-human mammals in which the stabilization of intracellular calcium ion signaling derangements and the inhibition of beta-secretase activity is desired. Advantages of this invention over alternatives (e.g. taking the two active ingredients separately in two separate dosage forms) include improved management of medications, a reduced pill burden, convenient dosing, simplified treatment, and improved adherence.

### DETAILED DESCRIPTION OF THE INVENTION

The terms “individual,” “subject,” and “patient,” are used interchangeably herein to refer to a mammal, and can encompass a human or a non-human mammal.

The singular forms “a,” “and,” and “the” as used herein include plural referents unless the context clearly dictates otherwise.

The term “single dosage form” is used herein to refer to a single dose wherein all active and inactive ingredients are combined in a suitable system, such that the patient or person administering the drug to the patient can open a single container or package with the entire dose contained therein. Typical examples of single dosage forms are tablets or capsules for oral administration, single dose vials for injection, or suppositories for rectal administration. This aforementioned list of single dosage forms is not intended to be limiting in any way, but merely to represent typical examples of single dosage forms.

The term “pharmaceutically acceptable carrier” is used herein to refer to a carrier that is useful in preparing a pharmaceutical composition that is generally safe, non-toxic and neither biologically nor otherwise-undesirable, and is acceptable for veterinary use as well as human pharmaceutical use. A “pharmaceutically acceptable carrier” as used in the specification and claims can include both one and more than one such carrier. By “pharmaceutically acceptable” it is meant the carrier must be compatible with the other ingredients of the formulation and not deleterious to the recipient thereof.

The terms “administration of” or “administering a” pharmaceutical composition should be understood to mean providing a pharmaceutical composition to an individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “treatment” or “treating” means any administration of a pharmaceutical composition of this invention to obtain a desired pharmacologic and/or physiologic effect. The effect may be prophylactic in terms of completely or partially preventing a disease or symptom thereof, and/or may be therapeutic in terms of a partial or complete cure for a disease and/or adverse affect attributable to the disease. Treatment includes (a) inhibiting the disease in the subject that is experiencing or displaying the pathology or symptomatology of the diseased (i.e., arresting further development of the pathology and/or symptomatology), or (b) ameliorating the disease in the subject that is experiencing or displaying the pathology or symptomatology of the diseased (i.e., reversing the pathology and/or symptomatology).

Pharmaceutical compositions of this invention may be prepared by any of the methods well known in the art of pharmacy.

Pharmaceutical compositions of this invention encompass any composition made by admixing the active ingredients and a pharmaceutically acceptable carrier. The carrier may take a wide variety of forms depending on the form of preparation desired for administration, e.g., oral or parenteral (including intravenous). Thus, the pharmaceutical composition of the invention can be presented as discrete units suitable for oral administration such as capsules, cachets or tablets each containing a predetermined amount of the active ingredients. Further, the composition can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the composition may also be administered by controlled release means and/or delivery devices. The foregoing list is illustrative only and is not intended to be limiting in any way.

Pharmaceutical compositions of this invention intended for oral use may contain one or more agents selected from the group consisting of sweetening agents, flavoring agents, coloring agents and preserving agents in order to provide pharmaceutically elegant and palatable preparations. Tablets may contain a composition of the invention in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. A tablet may be prepared by compression or molding, optionally with one or more accessory ingredients or adjuvants. Compressed tablets may be prepared by compressing, in a suitable machine, a compound of the invention in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of this invention for oral use may also be presented as hard gelatin capsules wherein the compound of the invention is mixed with an inert solid diluent, for example, calcium carbonate, calcium phosphate or kaolin, or as soft gelatin capsules wherein the compound of the invention is mixed with water or an oil medium, for example peanut oil, liquid paraffin, or olive oil.

Pharmaceutical compositions of this invention include aqueous suspensions, which contain the active materials in admixture with excipients suitable for the manufacture of aqueous suspensions. In addition, oily suspensions may be formulated by suspending the compound of the invention in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

Pharmaceutical compositions of this invention can be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and should be preserved against the contaminating action of microorganisms such as bacteria and fungi.

Pharmaceutical compositions of this invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound of the invention, to produce a cream or ointment having a desired consistency.

Pharmaceutical compositions of this invention can also be in a form suitable for rectal administration wherein the carrier is a solid. Suitable carriers include cocoa butter and other materials commonly used in the art.

“ Levetiracetam” (also known as “Keppra”), refers to any form of levetiracetam, or a derivative, analog, generic equivalent, pharmaceutically acceptable salt, hydrate, solvate, polymorph, or prodrug thereof; that is an active ingredient in a pharmaceutical composition as known in the art of pharmacy. Levetiracetam can refer to any type of long acting or extended release form of levetiracetam (e.g. “Keppra XR”), and/or any type of levetiracetam admixture (i.e. any type of pharmaceutical composition with any form or prodrug of levetiracetam added for the purpose of exerting pharmacological and physiological effects on a subject).

“Levetiracetam” as used in the specification and claims can include both one and more than one pharmaceutical composition containing levetiracetam.

Inosoitol triphosphate receptor channels (also known as “InsP3 receptor channels”, “IP3 receptor channels”, “IP3R channels”, “Inositol 1,4,5-triphosphate receptors”) are integrated within the endoplasmic reticulum of cells. Levetiracetam has been shown to inhibit calcium ion flow from the endoplasmic reticulum into the cytoplasm through inositol triphosphate receptor channels (see Nagarkatti N. et al., Neurosci. Lett., vol. 436, 2008, 289-293).

Ryanodine receptor channels (also known as “RyR channels”) are integrated within the endoplasmic reticulum of cells. Levetiracetam has been shown to inhibit calcium ion flow from the endoplasmic reticulum into the cytoplasm through ryanodine receptor channels (see Nagarkatti N. et al., Neurosci. Lett., vol. 436, 2008, 289-293).

Levetiracetam may influence the activity of substrates in addition to or other than inositol triphosphate receptor channels and ryanodine receptor channels, such as synaptic vesicle glycoprotein SV2A (see Vogl C. et al., Mol. Pharmacol., vol. 82, 2012, 199-208). The possible influence of levetiracetam on substrates in addition to or other than inositol triphosphate receptor channels and ryanodine receptor channels does not limit the intended use of this invention, which is for treating disease in which the stabilization of intracellular calcium ion derangements and the inhibition of beta-secretase activity is desired. “ Beta-secret a se” (known as “p-secretase”, “BACE”) refers to enzymes that are also known as p-site cleaving enzyme types 1 and 2 (“BACE1”, and “BACE2”—a homolog of BACE1) (see Vassar R. et al., Science, vol. 286, 1999, 735-741; Yan R. et al., Nature, vol. 402, 1999, 533-537; Bennett B. D. et al., J. Biol. Chem., 2000, vol. 275, 20647-20651; Farzan M. et al., 2000, Proc. Natl. Acad. Sci. U.S.A., vol. 97, 2000, 9712-9717; Vassar R. et al., J. Neurochem., vol. 130, 2014, 4-28). It is generally accepted that beta-secretase type 1 (i.e. BACE1) cleaves amyloid precursor protein at a location on amyloid precursor protein characterized as the beta-secretase cleavage site (see Querfurth H. W. and LaFerla F. M., N. Engl. J. Med., vol. 362, 2010, 329-344; Vassar R. et al., J. Neurochem., vol. 130, 2014, 4-28).

A beta-secretase inhibitor (known as a “BACE inhibitor”) is a compound that impedes the activity of beta-secretase (see Cole S. L. and Vassar R., Mol. Neurodegener., vol. 2, 2007, 22; Ghosh A. K. et al., Neurotherapeutics, vol. 5, 2008, 399-408; Querfurth H. W. and LaFerla F. M., N. Engl. J. Med., vol. 362, 2010, 329-344; Vassar R. et al., J. Neurochem., vol. 130, 2014, 4-28). This includes beta-secretase inhibitors either in or having been in clinical trials or preclinical trials (e.g. AZD3293, AZD3839, CNP520, CTS-21166, E2609, GRL-8234, GSK188909, HPP854, JNJ-54861911, LY2886721, LY2811376, MBI-3, NB-360, PF-05297909, RG7129, R05508887, SCH 1359113, SCH4549145, TAK-070, Verubecestat—also known as MK-8931); and/or any other compounds or molecules in which inhibition of beta-secretase activity has been determined and demonstrated by methodology known in the art. The foregoing list of beta-secretase inhibitors is illustrative only and is not intended to be limiting in any way.

A “beta-secretase inhibitor” as used in the specification and claims can include both one beta-secretase inhibitor and more than one beta-secretase inhibitor.

Beta-secretase types 1 and 2 (i.e. BACE1, BACE2) may have activity on substrates in addition to or other than amyloid precursor protein, and examples of candidate substrates include neurexin 1 alpha, peptidyl-amidating monooxygenase, type I transmembrane protein, premelanosome protein, seizure 6-like protein, and insulin-like growth factor 2 receptor (for an expanded list of candidate substrates of beta-secretase, see Vassar R. et al., J. Neurochem., vol. 130, 2014, 4-28). The possible effect of beta-secretase on substrates in addition to or other than amyloid precursor protein does not limit the intended use of this invention, which is for treating disease in which the stabilization of intracellular calcium ion derangements and the inhibition of beta-secretase activity is desired.

The present invention provides a pharmaceutical composition comprising of at least two active ingredients: (a) levetiracetam, and (b) a beta-secretase inhibitor; to treat, ameliorate, control, or reduce the risk of disease in which the stabilization of intracellular calcium ion derangements and the inhibition of beta-secretase activity is desired, so that this pharmaceutical composition will include at least:
(a) Levetiracetam, or a derivative, analog, generic equivalent, pharmaceutically acceptable salt, hydrate, solvate, polymorph, or prodrug thereof. Levetiracetam can refer to any type of long acting or extended release form of levetiracetam (e.g. “Keppra XR”), and/or any type of levetiracetam admixture (i.e. any type of pharmaceutical composition with any form or prodrug of levetiracetam added for the purpose of exerting pharmacological and physiological effects on a subject).
(b) a beta-secretase inhibitor or beta-secretase inhibitors; which can include a beta-secretase inhibitor either in or having been in clinical trials or preclinical trials (e.g. AZD3293, AZD3839, CNP520, CTS-21166, E2609, GRL-8234, GSK188909, HPP854, JNJ-54861911, LY2886721, LY2811376, MBI-3, NB-360, PF-05297909, RG7129, R05508887, SCH 1359113, SCH4549145, TAK-070, Verubecestat—also known as MK-8931); and/or generic equivalents, and/or any other compound or molecule in which inhibition of beta-secretase activity has been determined and demonstrated by methodology known in the art. The foregoing list of beta-secretase inhibitors is illustrative only and is not intended to be limiting in any way.

It is generally accepted that mild cognitive impairment represents an initial or prondromal stage of Alzheimer's disease, and that pharmaceutical agents that may prevent Alzheimer's disease might also prevent mild cognitive impairment (see Gauthier S. et al., Lancet., vol. 367, 2006, 1262-1270; Petersen R. C. et al., Arch Neurol., vol. 66, 2009, 1447-1455).

This invention relates to the manufacture or compounding of a pharmaceutical composition; for use in the treatment and prevention of mild cognitive impairment, Alzheimer's disease, and other conditions or diseases that may be treated and/or prevented by stabilizing intracellular calcium ion derangements and inhibiting beta-secretase activity; such as Down's syndrome, cerebrovascular dementia, progressive supranuclear palsy (“PSP”), other frontotemporal dementias, Lewy body dementia, Huntington's disease, Parkinson's disease, Parkinson's disease dementia, cerebral amyloid angiopathy (“CAA”), hereditary cerebral hemorrhage with amyloidosis of the Dutch type (“HCHWA-D”), cerebral autosomal dominant arteriopathy with subcortical infarcts and leukoencephalopathy (“CADASIL”), adult metochromatic leukodytrophy, Binzwanger disease, adult-onset adrenoleukodystrophy, other leukodystrophies, Creutzfeld-Jakob disease, prion disorders, HIV associated dementia (“HAD”), amyotrophic lateral sclerosis (“ALS”), spinocerebellar ataxia, head trauma, traumatic brain injury (“TBI”), chronic traumatic encephalopathy (“CTE”), stroke, genetic variants associated with an increased incidence and/or prevalence of dementia such as familial Alzheimer's disease mutations (e.g. mutations in amyloid precursor protein, presenilin 1, presenilin 2) and Apolipoprotein E (“ApoE4”) mutations, pancreatitis, diabetes, cardiovascular disease, seizure disorders, mood disorders, and atherosclerosis. The foregoing list is illustrative only and is not intended to be limiting in any way.

The subject or patient to whom the present invention is intended for is a human being, male or female; but may also encompass other mammals, such as dogs, cats, mice, rats, cattle, horses, sheep, rabbits, monkeys, chimpanzees or other apes or primates, in which the stabilization of intracellular calcium ion derangements and the inhibition of beta-secretase activity is desired.

Amounts of the component active ingredients in this invention may be varied to (a) provide an optimal therapeutic response and (b) to minimize side effects.

General parameters for the amount of levetiracetam in this invention may vary and may depend on FDA recommendations, the age, weight, and medical condition of the subject for whom this invention is intended, the dosing interval, and the pharmacodynamics and pharmacokinetics of the levetiracetam. The foregoing list of parameters for determining the amount of levetiracetam in this invention is illustrative only and is not intended to be limiting in any way.

General parameters for the amount of the beta-secretase inhibitor in this invention may vary and may depend on FDA recommendations, the age, weight, and medical condition of the subject for whom this invention is intended, the dosing interval, and the pharmacodynamics and pharmacokinetics of the beta-secretase inhibitor. The foregoing list of parameters for determining the amount of the beta-secretase inhibitor in this invention is illustrative only and is not intended to be limiting in any way.

General parameters for the dosing interval of this invention, and the time of day when this invention is to be administered, may vary and may depend on FDA recommendations, the age, weight, and medical condition of the subject for whom this invention is intended, and the pharmacodynamics and pharmacokinetics of the component active ingredients. The foregoing list of parameters for determining the dosing interval and the time of day for administering this invention is illustrative only and is not intended to be limiting in any way.

Those skilled in the art will understand that various modifications may be made to the invention without departing from the spirit or scope thereof. Thus, the present invention is intended to encompass all modifications and variations within the scope of the appended claims and their equivalents.

### REFERENCES CITED—OTHER PUBLICATIONS

Bennett B. D. et al., “Expression analysis of BACE2 in brain and peripheral tissues”, J. Biol. Chem., 2000, vol. 275, 20647-20651.

Berridge M. J., “Calcium signalling and Alzheimer's disease”, Neurochem. Res., vol. 36, 2011, 1149-1156.

Berridge M. J. et al., “Calcium—a life and death signal”, Nature, vol. 395, 1998, 645-648.

Bezprozvanny I. and Hiesinger P. R., “The synaptic maintenance problem: membrane recycling, Ca2+homeostasis and late onset degeneration”, Mol. Neurodegener., vol. 8, 2013, 23.

Bezprozvanny I. and Mattson M. P., “Neuronal calcium mishandling and the pathogenesis of Alzheimer's disease”, Trends Neurosci., vol. 31, 2008, 454-463.

Chakroborty S. et al., “Early calcium dysregulation in Alzheimer's disease: setting the stage for synaptic dysfunction”, Sci. China Life Sci., vol. 54, 2011, 752-762.

Cole S. L. and Vassar R., The Alzheimer's disease beta-secretase enzyme, BACE1″, Mol. Neurodegener., vol. 2, 2007, 22.

Danysz W. et al. “Neuroprotective and symptomatological action of memantine relevant for Alzheimer's disease—a unified glutamatergic hypothesis on the mechanism of action”, Neurotox. Res., vol. 2, 2000, 85-97.

De Caluwe J., Dupont G., “The progression towards Alzheimer's disease described as a bistable switch arising from the positive loop between amyloids and Ca(2+)”, J. Theor. Biol., vol. 331, 2013, 12-18.

Farzan M. et al., “BACE2, a beta-secretase homolog, cleaves at the beta site and within the amyloid-beta region of the amyloid-beta precursor protein”, Proc. Natl. Acad. Sci. U.S.A., vol. 97, 2000, 9712-9717.

Ferreira I. et al., “Amyloid beta peptide 1-42 disturbs intracellular calcium homeostasis through activation of GluN2B-containing N-methyl-d-aspartate receptors in cortical cultures”, Cell Calcium, vol. 51, 2012, 95-106.

Gauthier S. et al., “Mild cognitive impairment”, Lancet., vol. 367, 2006, 1262-1270.

Ghosh A. K. et al., “beta-Secretase as a therapeutic target for Alzheimer's disease”, Neurotherapeutics, vol. 5, 2008, 399-408.

Hardy J. and Allsop D., “Amyloid deposition as the central event in the aetiology of Alzheimer's disease”, Trends Pharmacol. Sci., vol. 12, 1991, 383-388.

Lopez J. R. et al., “Increased intraneuronal resting [Ca2+] in adult Alzheimer's disease mice”, J. Neurochem., vol. 105, 2008, 262-271.

Nagarkatti N. et al., “Levetiracetam inhibits both ryanodine and IP3 receptor activated calcium induced calcium release in hippocampal neurons in culture”, Neurosci. Lett., vol. 436, 2008, 289-293.

Petersen R. C. et al., “Mild cognitive impairment: ten years later”, Arch Neurol., vol. 66, 2009, 1447-1455.

Querfurth H. W. and LaFerla F. M., “Alzheimer's disease”, N. Engl. J. Med., vol. 362, 2010, 329-344.

Stutzmann G. E., “The pathogenesis of Alzheimers disease—is it a lifelong ‘ccalciumopathy’?”, Neuroscientist , vol. 13, 2007, 546-559.

Vassar R. et al., “Beta-secretase cleavage of Alzheimer's amyloid precursor protein by the transmembrane aspartic protease BACE”, Science, vol. 286, 1999, 735-741.

Vassar R. et al., “Function, therapeutic potential and cell biology of BACE proteases: current status and future prospects”, J. Neurochem., vol. 130, 2014, 4-28.

Vogl C. et al., “The synaptic vesicle glycoprotein 2A ligand levetiracetam inhibits presynaptic Ca2+ channels through an intracellular pathway”, Mol. Pharmacol., vol. 82, 2012, 199-208.

Wallace J., “Calcium dysregulation, and lithium treatment to forestall Alzheimer's disease—a merging of hypotheses”, Cell Calcium, vol. 55, 2014, 175-181.

Yan R. et al., “Membrane-anchored aspartyl protease with Alzheimer's disease beta-secretase activity”, Nature, vol. 402, 1999, 533-537.

I claim:

1. A pharmaceutical composition comprising of at least two active ingredients: (a) levetiracetam, in a therapeutically effective amount, and (b) a beta-secretase inhibitor, in a therapeutically effective amount, so that the pharmaceutical composition will include at least:
(a) Levetiracetam, or a derivative, analog, generic equivalent, pharmaceutically acceptable salt, hydrate, solvate, polymorph, or prodrug thereof. Levetiracetam can refer to any type of long acting or extended release form of levetiracetam (e.g. “Keppra XR”), and/or any type of levetiracetam admixture (i.e. any type of pharmaceutical composition with any form or prodrug of levetiracetam added for the purpose of exerting pharmacological and physiological effects on a subject); and
(b) a beta-secretase inhibitor or beta-secretase inhibitors; which can include a beta-secretase inhibitor either in or having been in clinical trials or preclinical trials (e.g. AZD3293, AZD3839, CNP520, CTS-21166, E2609, GRL-8234, GSK188909, HPP854, JNJ-54861911, LY2886721, LY2811376, MBI-3, NB-360, PF-05297909, RG7129, R05508887, SCH 1359113, SCH4549145, TAK-070, Verubecestat—also known as MK-8931); and/or generic equivalents, and/or any other compound or molecule in which inhibition of beta-secretase activity has been determined and demonstrated by methodology known in the art.

2. The pharmaceutical composition in claim 1, for use in the manufacture or compounding of a pharmaceutical medicament.

3. The pharmaceutical composition in claim 1, for use to treat, ameliorate, control, or reduce the risk of Alzheimer's disease.

4. The pharmaceutical composition in claim 1, for use to treat, ameliorate, control, or reduce the risk of Down's syndrome, cerebrovascular dementia, progressive supranuclear palsy (“PSP”), other frontotemporal dementias, Lewy body dementia, Huntington's disease, Parkinson's disease, Parkinson's disease dementia, cerebral amyloid angiopathy (“CAA”), hereditary cerebral hemorrhage with amyloidosis of the Dutch type (“HCHWA-D”), cerebral autosomal dominant arteriopathy with subcortical infarcts and leukoencephalopathy (“CADASIL”), adult metochromatic leukodytrophy, Binzwanger disease, adult-onset adrenoleukodystrophy, other leukodystrophies, Creutzfeld-Jakob disease, prion disorders, HIV associated dementia (“HAD”), amyotrophic lateral sclerosis (“ALS”), spinocerebellar ataxia, head trauma, traumatic brain injury (“TBI”), chronic traumatic encephalopathy (“CTE”), stroke, genetic variants associated with an increased incidence and/or prevalence of dementia such as familial Alzheimer's disease mutations (e.g. mutations in amyloid precursor protein, presenilin 1, presenilin 2) and Apolipoprotein E (“ApoE4”) mutations, pancreatitis, diabetes, cardiovascular disease, seizure disorders, mood disorders, and atherosclerosis; and any other condition or disease that may be treated, prevented, ameliorated, or controlled by stabilizing intracellular calcium ion derangements and inhibiting beta-secretase activity.

5. The pharmaceutical composition in claim 1, for use to treat, ameliorate, control, or reduce the risk of disease in non-human mammals.
